(19) United States
(12) Patent Application Publication
(10) Pub. No.: US 2003/0095959 A1
(43) Pub. Date: May 22, 2003

(54) Topical skin composition

(75) Inventor: James R. Mayne, Kentwood, MI (US)

Correspondence Address: BRINKS, HOFER, GILSON & LIONE, IN RE: ALTICOR INC., P.O. BOX 10395, CHICAGO, IL, 60610, US

(73) Assignee: ACCESS BUSINESS GROUP INTERNATIONAL LLC.

(21) Appl. No.: 10/155,305

(22) Filed: May 24, 2002

Related U.S. Application Data
(63) Continuation-in-part of application No. PCT/US00/31933, filed on Nov. 21, 2000.

(51) Int. Cl. A61K 38/44; A61K 31/555; A61K 31/409

(52) U.S. Cl. 424/94.4; 514/185; 514/410

(57) ABSTRACT

A topical skin composition that includes a complex containing an effective amount of selected components to provide a defense against the various pathway mechanisms of reactive oxygen species. In addition, a method for the treatment of the skin is provided. The composition and method are directed to the prevention of the adverse or detrimental effects of reactive oxygen species.

This application is a continuation-in-part application of PCT/US00/31933, the entire contents of which is incorporated herein.

### BACKGROUND OF THE INVENTION

With an aging population, there has been an increase in the study of aging as it relates to the human body. For example, the treatment of aging skin exhibited by the presence of fine lines, wrinkles, and the like has received a great deal of attention. The dermal signs of aging such as fine lines, wrinkles, laxity, and hyperpigmentation have been fought through many tactics including surgery, laser treatment and cosmetics. Cosmetic treatments include the use of various creams and lotions to alter the effects of dermal aging. Much of the literature in the prior art focuses on the use of a single primary ingredient to prevent one of several deleterious aging affects. For example, one tactic has been to use one or more hydroxy acids or retinoic acid to stimulate the re-growth of dermal cells, without other ingredients. This approach is flawed because it does not recognize that aging is caused by the deleterious interaction of multiple agents on the skin, from multiple sources, causing damage to the skin through multiple simultaneous damage pathways.

Another area that is receiving a great deal of attention is the effect of free radicals, reactive oxygen species (“ROS”), and other oxidizing species (“OOS”) on the human body including the skin. These entities have been implicated in a number of skin conditions including photodamage, general aging of the skin, contact dermatitis, wrinkling, inflammation, and damage to the skin tissue.

The ROS species include superoxide (O2—), hydrogen peroxide (H2O2), peroxy radicals (HO2 and RO2) alkyl peroxide (R2O2), hydroxyl radical (.OH), alkoxy radical (.OR), and singlet oxygen. The OOS species include hypohalous acids (HOX) (where X is chloride, bromide, iodide), Z-amines (where Z is either chlorinated or ammoniated amine containing compounds, nitric oxide (NO), ammonia, cyclooxygenase, phospholipase A2, phospholipase C and transition metals.

Each of the ROS directly or acting as an intermediate are thought to act on cell membrane to adversely impact the skin. Thus, there is a need for a topical skin treatment composition and method that provides a defense against each of the ROS. In addition, it would be desirable if such a composition repaired damage caused by the ROS.

### BRIEF SUMMARY OF THE INVENTION

The present invention is directed to a complex containing an effective amount of selected components to provide a defense against the various pathway mechanisms of ROS. The invention also contemplates a method for the treatment of the skin. Specifically, the composition and method of this invention are directed to the prevention of the adverse or detrimental effects of ROS.

The present invention also contemplates a composition that repairs damage caused by the ROS.

In general, the complex composition contains at least one anti-superoxide component, at least one anti-hydrogen peroxide component, at least one anti-hydroxyl radical component, and optionally at least one chain breaker component.

In one embodiment, the composition also contains a component that aids in cellular energy product, a component that aids in collagen synthesis, and a component that aids or provides cellular activity.

### BRIEF DESCRIPTION OF THE DRAWINGS

FIG. 1 is a graph showing the increase in erythema 30 minutes after UV exposure on human skin to which formulations according to the present invention were applied.

FIG. 2 is a graph showing the increase in erythema 10 hours after UV exposure on human skin to which formulations according to the present invention were applied

### DETAILED DESCRIPTION OF THE INVENTION

The present invention provides a topical skin complex composition that provides a defense mechanism against a variety of ROS.

In general, the complex composition contains at least one anti-superoxide component, at least one anti-hydrogen peroxide component, at least one anti-hydroxyl radical component, and optionally at least one chain breaker. In one embodiment, the composition also contains a component that aids in cellular energy product, a component that aids in collagen synthesis, and a component that aids or provides cellular activity.

The composition also includes a cosmetically or pharmaceutically acceptable carrier. When a carrier is present, the complex forms from about 0.01% to about 10% by weight of the total composition, preferably from about 1% to about 7% of the total composition. The particular components of the complex will now be described in more detail below.

Anti-Superoxide Component

In general this component includes those materials having anti-superoxide activity and, in particular, those having superoxide dismutase activity. In other words, it includes those components that can catalyze a dismutation reaction. For example, it includes superoxide dismutase (SOD), SODs modified by grafting polyalkylene oxide, polyethylene glycol, polysaccharide or acylated groups, salts of SOD, substances containing such SOD products, porphorins and materials with superoxide dismutase-like activity. In this respect, it includes those products mentioned in EP 223 257, the relevant contents of which are incorporated herein by reference.

All the superoxide dismustases described above, as well as the variants and equivalents that a person of skill in the art can deduce from the literature may be suitable as SODs for use in the present invention. In addition, they can be of differing origins.

For example, they may be animal (bovine, porcine, and the like), human (blood), or plant (fungi, algae, spinach, and the like). They may also be obtained from bacteria or yeast, or alternatively by a biotechnological route.

Examples of SODs that may have application in the present invention are described in U.S. Pat. No. 5,526,507, the contents of which is incorporated herein by reference.

The SOD forms from about 0.01% to about 5% by weight of the complex. Preferably, the SOD is included in the complex in an amount from about 0.1% to about 2% by weight.

Anti-Hydrogen Peroxide Component

In general, this component is a thiol or thiol derivative. In the context of the present invention, the term thiol is to be understood to be an organic compound characterized by the —SH group. Thiol derivatives are organic compounds that are either derivatives that retain the —SH group or are thio ethers or thio esters, in which case the —SH group is converted into the —SR group.

Compounds that are to be understood as being identical to the thiols or thiol derivatives according to the invention are those that are formed by tautomerism, di- or oligomerization by hydrogen bonding, hydration or other spontaneous rearrangement from the thiols or thiol derivatives. If a derivative is in equilibrium with an isomer by a different type of rearrangement, for example, migration of an alkyl group, this isomer is regarded as being included in the thiols and thiol derivatives of the invention.

Suitable thiol and thiol derivatives may include captopril, cysteamine, ergothioneine, mercaptopropionylglycine, penicillamine, N-acetylcysteine, S-acetylcysteine, N,S-diacetylcysteine, N,S-diacetylcysteinamide, cysteine ethyl ester, N-acetylcrysteine ethyl ester, S-acetylcysteine ethyl ester, N,S-diacetylcysteine ethyl ester, thioglycolic acid, cysteine, homocysteine, glutathione, thioglycerol, thiomalic acid, 2-mercaptopropionic acid, 3-mercaptopropionic acid, thiodiglycol, 2-mercaptoethanol, dithioreitol, thioxanthene, thiosalicylic acid, thiolactic acid, thiopropionic acid, thiodiglycolic acid, lipoic acid, and cosmetically acceptable salts thereof.

As used herein, the cosmetically acceptable salts include, but are not limited to alkali metal salts, e.g., sodium, lithium, potassium, and rubidium salts; alkaline earth metal salts, e.g. magnesium, calcium, and strontium salts; non-toxic heavy metal salts, e.g., aluminum and zinc salts; boron salts; silicon salts; ammonium salts; trialkylammonium salts, e.g. trimethylammonium and triethylammonium, and tetraalkylonium salts.

The anti-hydrogen peroxide component is incorporated into the complex in an amount from about 0.001% to about 5% by weight, preferably from about 0.01% to about 2.5%, more preferably from about 0.1% to about 1% by weight of the complex.

Anti-Hydroxyl Radical Component

The anti-hydroxyl radical component can include one or more of the following: tocopherol, tocopherol derivatives, tetrahydrodiferuloylmethane, grape seed extract, green tea extract, turmeric acid, curcuminoids, tetrahydrocurcuminoids catechins, epigallocatechin 3-0-gallate and polyphenols, oligomeric proanthocyanidins, bioflavonoids, flavonoids, and mixtures thereof.

Tocopherol (Vitamin E) and its derivatives such as esters of tocopherol are useful in the composition of the present invention. Suitable tocopherols include the monomethyl, dimethyl, or triethyl derivatives of tocol, including but not limited to, alpha tocopherol, beta tocopherol, gamma tocopherol, delta tocopherol, epsilon tocopherol, zeta tocopherol, and eta tocopherol. Suitable esters of tocopherol include but are not limited to tocopheryl acetate, tocopheryl succinate, tocopheryl benzoate, tocopheryl propionate, tocopheryl sorbate, tocopheryl oleate, tocopheryl orotate, tocopheryl linoleate, tocopheryl nicotinate, and the 2-ethyl-hexanoate ester.

When the tocopherol or its derivatives are included in the complex of the present invention, they are used at level from about 10% to about 98%.

Tetrahydrodiferuloylmethane and/or turmeric extract may also be incorporated into the complex at levels from about 0.1% to about 20% by weight of the complex, preferably from about 1% to about 10% by weight.

Grape seed extract and complexes of grape seed extract with phospholipids may also be beneficial for use in the present invention. The extracts from grape seed include a mixture polyphenols such as epicatechin, proanthocyanidins, and catechins. A suitable complex of grape seed extract and phospholipid is described in U.S. Pat. No. 4,963,527, the contents of which is incorporated herein by reference.

When incorporated into the complex, the grape seed extract or its complex with phospholipids is present in an amount from about 0.001% to about 5% by weight of the complex, preferably from about 0.01% to about 2.5% by weight.

Green tea extract may be included in the same amounts as the grape seed extract.

Flavonoids and bioflavonoids may also be useful in the present invention. It has been reported in Bravo, Polyphenols: Chemistry, Dietary Sources, Metabolism, and Nutritional Significance, Nutrition Reviews, Vol. 56, No. 11, 317-33 (November, 1998), the contents of which are incorporated herein by reference, that flavonoids may be subdivided into 13 classes shown below:

| Flavonoid | Basic Structure |
|---|---|
| Chalcones | 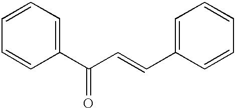 |
| Dihydrochalcones | 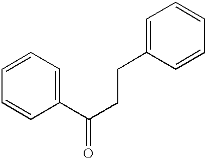 |
| Aurones | 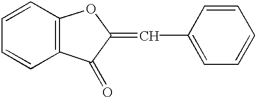 |
| | 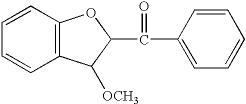 |
| Flavones | 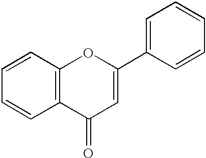 |
| Flavonols | 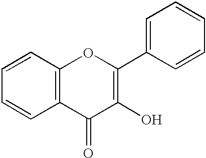 |
| Dihydroflavonol | 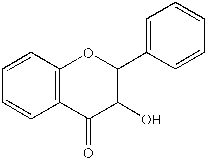 |
| Flavanones | 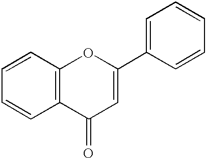 |
| Flavanol | 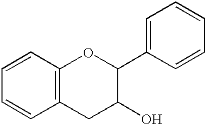 |
| Flavandiol or: leucoanthocyanidin | 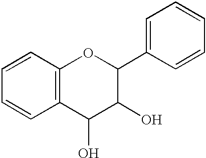 |
| Anthocyanidin | 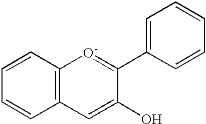 |
| Isoflavonoids | 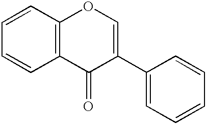 |
| | 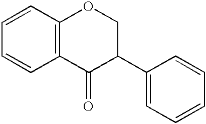 |
| Biflavonoids | 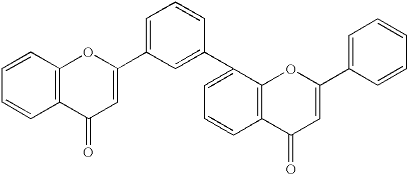 |
| Proanthocyanidins or condensed tannins | 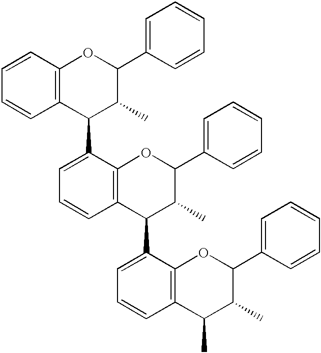 |

Flavonoids have, in general, the common structure of diphenylpropanes (C6-C3-C6) and consist of two aromatic rings linked through three carbons that usually form an oxygenated heterocycle. The basic structure is shown below:

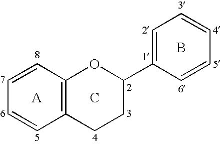

Flavonoids occasionally occur in plants as aglycones, although they are most commonly found as glycoside derivatives.

Specific suitable flavonoids for use in the present invention include but are not limited to rutin, citrin, quercitin, hesperidin, naringen, taxifolin, catechin, epicatechin, eriodictyol, naringenin, troxerutin, chrysin, tangeretin, luteolin, epigallocatechin, epigallocatechin gallate, fisetin, kaempferol, galangin, gallocetechin, epicatechin gallate, apigenin, diosmetin, myricetin, genistein, daidzein, or derivatives therof.

The flavonoids may be derived from any suitable source. A preferred source is from citrus.

When flavonoids are incorporated into the complex, they are present at a level from about 0.001% to about 20% by weight of the complex, preferably from about 0.01% to about 10% by weight.

Other specialty ingredients may also be included such as palmitoyl hydroxypropyltrimonium amylopectin. In one embodiment, the palmitoyl hydroxypropyltrimonium amylopectin can be mixed with camellia sinensis extract. This may be present in amounts ranging from about 0.001% to about 2% by weight of the complex.

Chain Breaker Component

The chain breaker may include the same components as those described above for the anti-hydroxyl radical component. Thus, one or more of the above anti-hydroxyl radical components may also serve as a chain breaker component. Chain breaking antioxidants are those ingredients that can break the chain reaction once lipid peroxidation is initiated.

As noted above, the complex composition may also include components selected to repair the damage caused by the ROS. In one embodiment, the compositions of the present invention includes at least one component that provides cellular energy production, at least one component that aids collagen synthesis, and at least one component that aids or provides cellular activity. These components may be used singly or, desirably, in combination.

Cellular Energy Production Component

A desirable cellular energy production component includes the ubiquinones. Ubiquinones are widely found in bacteria, fungi, yeasts, plants, and animals. It is known that different species produce isoforms (Q-n) with different numbers of isoprene units (n). For example, the number of isoprene units is 6 (Q6) in some microorganisms, nine (Q9) in plants, and ten (Q10) in humans. Coenzyme Q10 or 2,3,-dimethoxy-5-methyl-6-decaprenyl-benzoquinone functions to recover and maintain respiration and promotes ATP production in terms of energy supply for cellular activities. Derivatives of the ubiquinones such as ubiquinols may also be useful

The cellular energy production component, for example, coenzyme Q10, is incorporated into the complex in an amount ranging from about 0.001% to about 10%, preferably from about 0.01% to about 5% by weight of the complex.

Collagen Synthesis Component

To repair damage caused by ROS, it is desirable to include a component that will promote collagen synthesis. It has been suggested that hydroxy acids including alpha and beta hydoxy acids may be useful in this regard. As a result, the present invention contemplates including one or more alpha or beta hydroxy acids. Suitable examples include lactic, malic, glycolic, citric, and salicylic acid.

In addition, it has been found that ascorbic acid (Vitamin C) and its derivatives promote collagen synthesis. The ascorbic acid derivative useful in the present invention includes all enantiomers whether singly or in combination. Preferably, the ascorbic acid is provided in the levo form. In addition, the ascorbic acid or its derivatives may be in a water soluble or an oil soluble form.

Non-exclusive examples of the vitamin C (ascorbic acid) derivatives are, for instance, the alkyl esters of L-ascorbic acid where the alkyl portion has from 8 to 20 carbon atoms. With respect to the esters, they may be selected from the group consisting of fatty acid mono-, di-, tri- or tetra-esters of ascorbic acid. For example, such esters include, but are not limited to ascorbyl palmitate, ascorbyl laureate, ascorbyl myristate, ascorbyl stearate, ascorbyl dipalmitate, ascorbyl dilaurate, ascorbyl dimyristate, ascorbyl distearate, ascorbyl tripalmitate, ascorbyl trilaurate, ascorbyl trimyristate, ascorbyl tristearate, ascorbyl tetrapalmitate (tetrahexyldecyl ascorbate), ascorbyl tetralaurate, ascorbyl tetramyristate, ascorbyl tetrastearateL-ascorbyl palmitate, L-ascorbyl isopalmitate, L-ascorbyl dipalmitate, L-ascorbyl isostearate, L-ascorbyl distearate, L-ascorbyl diisostearate, L-ascorbyl myristate, L-ascorbyl isomyristate, L-ascorbyl 2-ethylhexanoate, L-ascorbyl di-2-ethylhexanoate, L-ascorbyl oleate and L-ascorbyl dioleate, tetrahexyl decyl ascorbate; phosphates of L-ascorbic acid such as L-ascorbyl-2-phosphate and L-ascorbyl-3-phosphate; sulfates of L-ascorbic acid such as L-ascorbyl-2-sulfate and L-acorbyl-3-sulfate; their salts with alkaline earth metals such as calcium and magnesium.

With respect to the salts, they may be selected from the phosphates and sulfates, preferably phosphate. The ascorbic acid phosphate is generally selected from L-ascorbic acid 3-phosphate, L-ascorbic acid 2-phosphate, L-ascorbic acid 3-pyrophosphate and bis (L-ascorbic acid 3,3-) phosphate. Preferably, the ascorbic acid phosphate is magnesium or sodium ascorbyl phosphate; more preferably, magnesium ascorbyl phosphate. Likewise, the ascorbic acid sulfate is generally selected from L-ascorbic acid 3-sulfate, L-ascorbic acid 2-sulfate, L-ascorbic acid 3-pyrosulfate and bis (L-ascorbic acid 3,3-) sulfate.

The collagen synthesis component, for example, the ascorbic acid and its derivatives, is incorporated in the complex in an amount ranging from about 0.001% to about 10%, preferably from about 0.01% to about 5% by weight of the complex.

Cellular Activity Component

It is believed that retinoids may affect cellular activity and thus it is desirable to incorporate retinoids in the complex of the present invention. The retinoids include retinol, retinal (Vitamin A aldehyde), and retinyl esters such as retinyl acetate, retinyl butyrate, retinyl propionate, retinyl octanoate, retinyl laurate, retinyl palmitate, retinyl oleate, and retinyl linoleate.

Retinoids tend to irritate the skin and therefore, it is desirable to incorporate them in the complex at levels so as to minimize the potential irritation. Alternatively, irritancy mitigants may be incorporated into the compositions to assist in preventing undue discomfort to the user while potentially permitting the dosage level of retinoid to be increased. Such irritancy mitigants include, but are not limited to ceramides, pseudoceramides, fatty acids, cholesterol, phospholipids, panthenol, oat extract, allantoin, ginkgo biloba, licorice extract, calendula, ginseng, butchers broom, and the like.

The cellular activity component, for example, the retinoid, is incorporated in the complex at a level ranging from about 0.001% to about 10%, preferably from about 0.01% to about 5% by weight of the complex.

The complex compositions according to the present invention are generally mixed with a pharmaceutically or cosmetically acceptable vehicle or carrier. The complex compositions of the present invention may be formulated as a solution, gel, lotion, cream, ointment, oil-in-water emulsion, water-in-oil emulsion, or other pharmaceutically or cosmetically acceptable form. The complex compositions of the present invention may also contain various known and conventional cosmetic ingredients so long as they do not detrimentally affect the desired effects.

The pharmaceutically acceptable or cosmetically acceptable vehicle acts as a dilutant, dispersant, or carrier for other materials present in the complex composition, so as to facilitate their distribution when the complex composition is applied to the skin.

Vehicles other than water can include liquid or solid emollients, solvents, humectants, thickeners, and powders. For example, the following vehicles can be used alone or as a combination of one or more vehicles.

Vehicles may also include propellants such as propane, isobutane, dimethyl ether, carbon dioxide, nitrous oxide; and solvents such as ethyl alcohol, isopropanol, acetone, ethylene glycol monomethyl ether, diethylene glycol monobutyl ether, diethylene glycol monoethyl ether, or powders such as chalk, talc, fullers earth, kaolin, starch, gums, collodial silica, sodium polyacrylate, tetra alkyl and/or trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate.

Emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rapeseed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate.

As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable materials.

The composition can optionally comprise suncreens such as inorganic and organic sunscreens to provide protection from the harmful effects of excessive exposure to sunlight during use of the complex composition of the present invention. Examples of suitable sunscreens include those described in the U.S. OTC Sunscreen Monograph, the contents of which is incorporated herein by reference.

The composition of the invention can accordingly comprise from 0.1 to 10%, preferably from 1 to 5% by weight of an organic sunscreen material.

The composition optionally can also comprise as a sunscreen titanium dioxide or zinc oxide having an average particle size of from 1 to 300 nm, iron oxide having an average particle size of from 1 to 300 nm, silica, such as fumed silica having an average particle size of from 1 to 100 nm. It should be noted that silica, when used as an ingredient in the emulsion according to the invention can provide protection from infrared radiation.

Ultrafine titanium dioxide in either of two forms, namely water-dispersible titanium dioxide and oil-dispersible titanium dioxide may be used. Water-dispersible titanium dioxide is ultrafine titanium dioxide, the particles of which are uncoated or which are coated with a material to impart a hydrophilic surface property to the particles. Examples of such materials include aluminum oxide and aluminum silicate. Oil-dispersible titanium dioxide is ultrafine titanium dioxide, the particles of which exhibit a hydrophobic surface property, and which, for this purpose, can be coated with metal soaps such as aluminum stearate, aluminum laurate, or zinc stearate, or with organosilicone compounds.

By “ultrafine titanium dioxide” is meant particles of titanium dioxide having an average particle size of less than 100 nm, preferably from 10 to 40 nm and most preferably from 15 to 25 nm. The total amount of titanium dioxide that can optionally be incorporated in the composition according to the invention is from 1 to 25%, preferably from 2 to 10% and ideally from 3 to 7% by weight of the composition.

Optional Skin Benefit Materials and Cosmetic Adjuncts

A particularly convenient form of the composition is an emulsion, in which case an oil or oily material (emollient) will normally be present, together with an emulsifier to provide either a water-in-oil emulsion or an oil-in-water emulsion.

The composition can also comprise water, usually up to 95%, preferably from 5 to 95% by weight.

Silicone Surfactant

The composition can also optionally comprise a high molecular weight silicone surfactant that can also act as an emulsifier, in place of or in addition to the optional emulsifier(s) already mentioned.

The silicone surfactant may be a high molecular weight polymer of dimethyl polysiloxane with polyoxethylene and/or polyoxpropylene side chains having a molecular weight of from 10,000 to 50,000. When used, the dimethyl polysiloxane polymer is conveniently provided as a dispersion in a volatile siloxane, the dispersion comprising, for example, from 1 to 20% by volume of the polymer and from 80 to 99% by volume of the volatile siloxane. Ideally, the dispersion consists of a 10% by volume of the polymer dispersed in the volatile siloxane.

Examples of the volatile siloxanes in which the polysiloxane polymer can be dispersed include polydimethyl siloxane (pentamer and/or hexamer).

A preferred silicone surfactant is cyclomethicone and dimethicone copolyol, such as DC 3225C Formulation Aid available from DOW CORNING. Another is laurylmethicone copolyol, such as DC Q2-5200, also available from Dow Corning.

The amount of silicone surfactant, when present in the composition will normally be up to 25%, preferably from 0.5 to 15% by weight of the emulsion.

Other Cosmetic Adjuncts

Examples of conventional adjuncts which can optionally be employed include preservatives, such as para-hydroxy benzoate esters; antioxidants, such butyl hydroxy toluene; humectants, such as glycerol, ethoxylated glycerins such as glycereth-26, sorbitol, 2-pyrrolidone-5-carboxylate, dibutylphthalate, gelatin, polyethylene glycol, such as PEG 200-600; buffers together with a base such as triethanolamine or sodium hydroxide; waxes, such as beeswax, ozokerite wax, paraffin wax; plant extracts, such as Aloe Vera, cornflower, witch hazel, elderflower, cucumber; as well as acerola cherry fermentate, thickeners; activity enhancers; colorants; and perfumes. Cosmetic adjuncts can form the balance of the composition.

It may also be desirable to incorporate anti-inflammatory and/or anti-irritant agents. The natural anti-inflammatory and/or anti-irritant agents are preferred. For example, licorice and its extracts, dipotassium glycyrrhizinate, oat and oat extracts, candelilla wax, alpha bisabolol, aloe vera, Manjistha (extracted from plants in the genus Rubia, particularly Rubia cordifolial), and Guggal (extracted from plants in the genus Commiphora, particularly Commiphora Mukul), may be used.

Skin conditioning agents such hyaluronic acid, its derivatives and salts including sodium hyaluronate, plant extracts such as kola nut, guarana mate, algae extract and skin benefit agents such as ceramides, glycoceramides, pseudoceramides, sphingolipids such as sphingomyelins, cerebrosides, sulphatides, and ganglioside, sphingosines, dihydrosphingosine, phytosphingosines, phospholipids, may also be incorporated, either separately or in mixtures. Fatty acids may also be combined with these skin benefit agents. For example, the ceramides and glycoceramides include those described in U.S. Pat. Nos. 5,589,178, 5,661,118, and 5,688,752, the relevant portions of which are incorporated herein by reference. For example, the pseudoceramides include those described in U.S. Pat. Nos. 5,198,210; 5,206,020; and 5,415,855, the relevant disclosures of which are incorporated herein by reference.

The following examples illustrate, but do not limit, the present invention. Unless otherwise indicated, all parts and percentages are by weight.

### EXAMPLE 1

The following is a topical skin composition according to the present invention.

| Ingredient | Wt. % |
|---|---|
| D. I. Water | 56.595 |
| Anti-superoxide component (superoxide dismutase) | 0.005 |
| Anti-hydrogen peroxide component (glutathione) | 0.2 |
| Anti-hydroxyl radical component (tocopheryl acetate) | 1.0 |
| Anti-hydroxyl radical component (tocopherol) | 0.2 |
| Anti-hydroxyl radical component (Tetrahydrodiferuloylmethane) | 0.1 |
| Anti-hydroxyl radical component (Grape (Vitis Vinifera) Seed | 0.1 |
| Extract (&) Phospholipids) | |
| Anti-hydroxyl radical component (Bioflavonoids) | 0.1 |
| Anti-hydroxyl radical component (Palmitoyl | 0.1 |
| Hydroxypropyltrimonium Amylopectin/Glycerin Crosspolymer | |
| (and) Lecithin (and) Camellia Sinensis Extract) | |
| Emollient(s) | 21.5 |
| Humectant(s) | 5.205 |
| Emulsifier(s) | 2.3 |
| Skin conditioning agent(s) | 0.1 |
| Sunscreen(s) (UVA) | 3.0 |
| Sunscreen(s) (UVB) | 7.5 |
| Thickener(s) | 0.3 |
| pH modifier(s) | 0.3 |
| Preservative(s) | 1.25 |
| Fragrance(s) | 0.1500 |
| TOTAL | 100.000 |

### EXAMPLE 2

The following is a topical skin composition according to one embodiment of the present invention. In this embodiment, the composition provides a defense against ROS and also includes ingredients to help repair damage caused ROS.

| Ingredient | Wt % |
|---|---|
| D. I . Water | 57.635 |
| Anti-superoxide component (superoxide dismutase) | 0.005 |
| Anti-hydrogen peroxide component (glutathione) | 0.2 |
| Anti-hydroxyl radical component (tocopheryl acetate) | 1.0 |
| Anti-hydroxyl radical component (tocopherol) | 0.2 |
| Anti-hydroxyl radical component (Tetrahydrodiferuloylmethane) | 0.1 |
| Anti-hydroxyl radical component (Grape (Vitis Vinifera) Seed | 0.1 |
| Extract (&) Phospholipids) | |
| Anti-hydroxyl radical component (Bioflavonoids) | 0.1 |
| Anti-hydroxyl radical component (Palmitoyl | 0.1 |
| Hydroxypropyltrimonium Amylopectin/Glycerin Crosspolymer | |
| (and) Lecithin (and) Camellia Sinensis Extract) | |
| Cellular activity component (retinyl acetate) | 0.16 |
| Cellular energy production component (Ubiquinone) | 0.05 |
| Collagen synthesis component (tetrahexyldecyl ascorbate) | 0.1 |
| Emollients | 26.5 |
| Humectants | 5.3 |
| Emulsifiers | 2.3 |
| Skin conditioning agent(s) | 0.1 |
| Silica (12 micron) | 2.0 |
| Silica (3 micron) | 2.0 |
| Aloe vera gel | 1.0 |
| Thickener(s) | 0.3 |
| pH modifier(s) | 0.3 |
| Preservative(s) | 0.3 |
| Fragrance | 0.150 |
| TOTAL | 100.00 |

### EXAMPLE 3

The following is a topical skin composition according to one embodiment of the present invention. In this embodiment, the composition provides a defense against ROS and also includes ingredients to help repair damage caused ROS.

| Ingredient | Wt. % |
|---|---|
| D.I. Water | 66.68 |
| Anti-superoxide component (superoxide dismutase) | 0.005 |
| Anti-hydrogen peroxide component (glutathione) | 0.2 |
| Anti-hydroxyl radical component (tocopheryl acetate) | 1.0 |
| Anti-hydroxyl radical component (tocopherol) | 0.2 |
| Anti-hydroxyl radical component (Tetrahydrodiferuloylmethane) | 0.1 |
| Anti-hydroxyl radical component (Grape (Vitis Vinifera) Seed | 0.1 |
| Extract (&) Phospholipids) | |
| Anti-hydroxyl radical component (Bioflavonoids) | 0.1 |
| Anti-hydroxyl radical component (Palmitoyl | 0.1 |
| Hydroxypropyltrimonium Amylopectin/Glycerin Crosspolymer | |
| (and) Lecithin (and) Camellia Sinensis Extract) | |
| Cellular activity component (retinyl acetate) | 0.16 |
| Cellular energy production component (Ubiquinone) | 0.05 |
| Collagen synthesis component (tetrahexyldecyl ascorbate) | 0.1 |
| Emollients | 1 |
| Humectants | 1.65 |
| Emulsifiers | |
| Skin conditioning agent(s) | 0.1 |
| Thickener(s) | 0.2 |
| pH modifier(s) | |
| Preservative(s) | 0.3 |
| Fragrance | 0.15 |
| Cyclomethicone | 10.00 |
| Polyglycerylmethacrylate | 10.00 |
| Dimethicone Copolyol | 2.00 |
| 12 micron Silica | 2.00 |
| 3 micron Silica | 2.00 |
| Polyacrylamide (and) C₁₃₋₁₄ Isoparaffin (and) Laureth-7 | 1.00 |
| Polysorbate 20 | 0.50 |
| TOTAL | 100.00 |

### EXAMPLE 4

The following tests were performed to determine the effect of providing a complex composition according to the present invention in comparison to a placebo, Vitamin E, and Vitamin C. The tests were conducted by outlining a number of two inch sections on the back of a human. The following formulas were applied in a randomized manner to the sections.

| | Wt. % | Wt. % | Wt. % | Wt. % |
|---|---|---|---|---|
| Ingredient | A | B | C | D |
| Emollient(s) | 21.5 | 21.5 | 21.5 | 21.5 |
| Humectant(s) | 6.205 | 6.205 | 6.205 | 6.205 |
| Emulsifier(s) | 1.3 | 1.3 | 1.3 | 1.3 |
| Skin conditioning agent(s) | 0.1 | 0.1 | 0.1 | 0.1 |
| Thickener(s) | 0.3 | 0.3 | 0.3 | 0.3 |
| pH modifier(s) | 0.3 | 0.3 | 0.3 | 0.3 |
| Preservative(s) | 1.25 | 1.25 | 1.25 | 1.25 |
| Fragrance(s) | 0.15 | 0.15 | 0.15 | 0.15 |
| Tocopherol | | 1.2000 | | 0.2000 |
| Glutathione | | | | 0.2000 |
| Tetrahydrodiferuloylmethane | | | | 0.1000 |
| Grape (Vitis Vinifera) Seed | | | | 0.1000 |
| Extract (&) Phospholipids | | | | |
| Bioflavonoids | | | | 0.1000 |
| Palmitoyl Hydroxypropyl- | | | | 0.1000 |
| trimonium Amylopectin/Glycerin | | | | |
| Crosspolymer (and) Lecithin (and) | | | | |
| Camellia Sinensis Extract | | | | |
| Superoxide dismutase | | | | 0.0050 |
| Sodium Hyaluronate | 0.0005 | 0.0005 | 0.0005 | 0.0005 |
| Ascorbic acid | | | 10.000 | |
| Water | QS | QS | QS | QS |

After the above formulations were applied, the back was subjected to UV radiation and the skin erythema was measured.

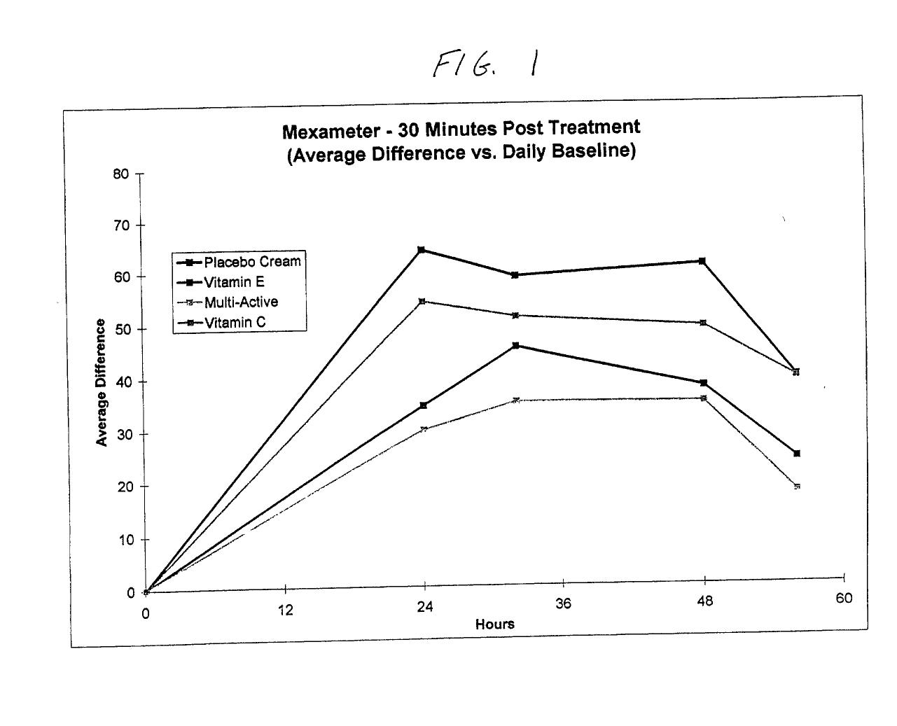

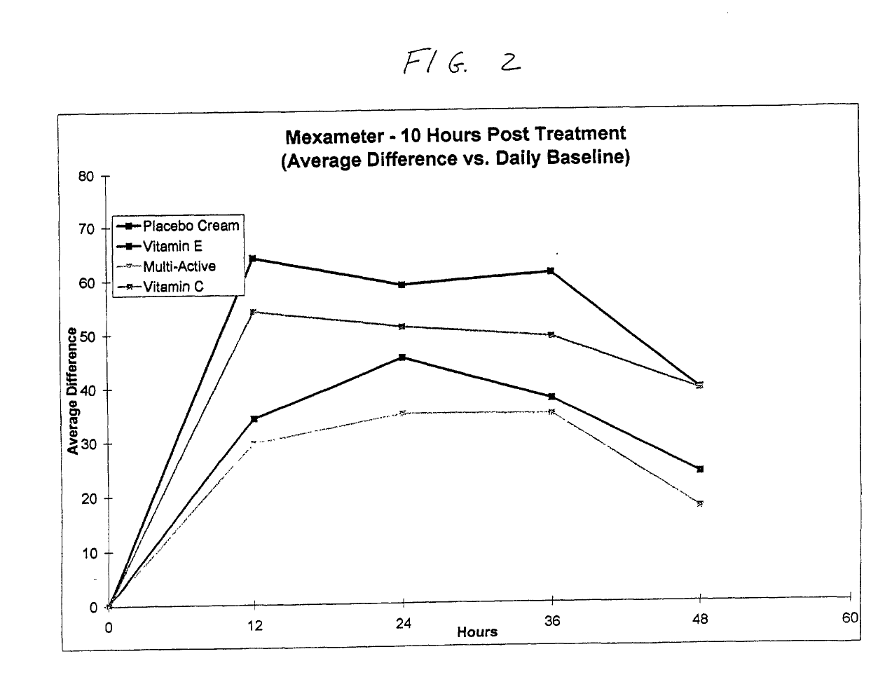

FIGS. 1 and 2 show the results.

It should be understood that a wide range of changes and modifications could be made to the embodiments described above. It is therefore intended that the foregoing description illustrates rather than limits this invention, and that it is the following claims, including all equivalents, which define this invention.

What is claimed is:

1. A topical skin composition for topical treatment for the prevention and repair of damage caused by reactive oxygen species, the composition comprising:
a. a cosmetically acceptable vehicle; and
b. a therapeutically effective amount of
i. at least one anti-superoxide component;
ii. at least one component selected from the group consisting of an anti-hydrogen peroxide radical component and an anti-peroxyl radical component; and,
iii. at least one anti-hydroxyl radical component; wherein each of the components are present in an amount to counteract the reactive oxygen species reaction involving superoxide, hydrogen peroxide, and hydroxyl reactions.

2. The skin composition of claim 1 further including a component that aids in cellular energy production.

3. The skin composition of claim 1 further including a component that aids in collagen synthesis.

4. The skin composition of claim 1 further including a component that aids cellular activity.

5. The skin composition of claim 1 wherein the anti-superoxide component is selected from the group consisting of superoxide dismutase, derivatives of superoxide dismutase, porphorins, and mixtures thereof.

6. The skin composition of claim 1 wherein the anti-hydrogen peroxide component is a thio compound selected from the group consisting of captopril, cysteamine, ergothioneine, mercaptopropionylglycine, penicillamine, N-acetylcysteine, S-acetylcysteine, N,S-diacetylcysteine, N,S-diacetylcysteinamide, cysteine ethyl ester, N-acetylcrysteine ethyl ester, S-acetylcysteine ethyl ester, N,S-diacetylcysteine ethyl ester, thioglycolic acid, cysteine, homocysteine, glutathione, thioglycerol, thiomalic acid, 2-mercaptopropionic acid, 3-mercaptopropionic acid, thiodiglycol, 2-mercaptoethanol, dithioreitol, thioxanthene, thiosalicylic acid, thiolactic acid, thiopropionic acid, thiodiglycolic acid, lipoic acid, and cosmetically acceptable salts thereof.

7. The skin composition of claim 1 wherein the anti-hydroxyl radical is selected from the group consisting of tocopherol, tocopheryl esters, tocopherol acids, tocopherol salts, tetrahydroferuloylmethane, grape seed extract, green tea extracts, flavonoids, and mixtures thereof.

8. The skin composition of claim 2 wherein the component that aids in cellular energy production is selected from the group consisting of ubiquinones, ubiquinols, and mixtures thereof.

9. The skin composition of claim 3 wherein the component that aids in collagen synthesis is selected from the group consisting of ascorbic acid and its derivatives.

10. The skin composition of claim 10 wherein the ascorbic acid is selected from the group consisting of a water soluble ascorbic acid, a fat soluble ascorbic acid, derivatives of each, and mixtures thereof.

11. The skin composition according to claim 10 wherein the fat-soluble ascorbic acid is a tetra-ester of ascorbic acid.

12. The skin composition of claim 4 wherein the component that aids cellular activity is a retinoid.

13. The skin composition of claim 1 further comprising a skin growth promoter selected from the group consisting of hydroxy acids.

14. The skin composition of claim 13 wherein the skin growth promoter is an α-hydroxy acid.

15. The skin composition of claim 13 wherein the skin growth promoter is a β-hydroxy acid.

16. The skin composition of claim 1 further comprising a UV damage preventer.

17. The skin composition of claim 1 further comprising an anti-inflammatory.

18. The skin composition of claim 1 wherein the anti-hydrogen peroxide radical component is not methional, malondialdehyde, or a factor influencing the intracellular concentration of methional or malondialdehyde.

19. The skin composition of claim 1 wherein the cosmetically acceptable vehicle is in liquid form and is selected from the group consisting of a solution, gel, lotion, cream, ointment, oil-in-water emulsion, or water-in-oil emulsion.
